(19) United States
(12) Patent Application Publication
(10) Pub. No.: US 2009/0137055 A1
(43) Pub. Date: May 28, 2009

(54) MEASURING NITROGEN OXIDES AND OTHER GASES BY OZONE FORMATION

(76) Inventor: John A. Bognar, Bozeman, MT (US)

Correspondence Address: SHERIDAN ROSS PC, 1560 BROADWAY, SUITE 1200, DENVER, CO, 80202, US

(21) Appl. No.: 12/067,976

(22) PCT Filed: Sep. 26, 2006
(86) PCT No.: PCT/US2006/037693

§ 371 Date: Sep. 24, 2008

Related U.S. Application Data
(60) Provisional application No. 60/722,306, filed on Sep. 30, 2005.

(51) Int. Cl. G01N 21/00; G01N 33/00; A61B 5/08

(52) U.S. Cl. 436/118; 422/83; 600/532

(57) ABSTRACT

A photochemical sensing system enables the measurement of nitrogen oxides (nitrogen dioxide and nitric oxide) by photolyzing nitrogen dioxide to form oxygen atoms which combine with oxygen molecules to form ozone. Ozone reacts with nitric oxide to for nitrogen dioxide-decreasing ozone. Changes in ozone concentration are measured as a surrogate for the nitrogen dioxide and nitric oxide. Any species which photolyzes to yield oxygen atoms may be measured by this technique. Additional specificity for nitrogen oxides is conferred by allowing the nitric oxide to react with the ozone to recreate the nitrogen dioxide. By periodically photolyzing the nitrogen dioxide (to form ozone), and then allowing the resulting nitric oxide to react with the ozone (thereby reducing ozone), a pulsed signal is obtained whose amplitude is proportional to the total amount of nitrogen dioxide and nitric oxide present. Medical applications include measuring nitric oxide concentrations in expired air samples.

### RELATED APPLICATION DATA

This application claims the benefit of and priority under 35 U.S.C. § 119(e) to U.S. Patent Application No. 60/722,306, filed Sep. 30, 2005, entitled “Method and Apparatus to Measure Nitrogen Oxides and Other Gases by Ozone Formation,” which is incorporated herein by reference in its entirety.

### BACKGROUND

1. Field of the Invention

An exemplary embodiment of the invention is related to measurement of nitrogen oxides. More specifically, an exemplary embodiment of the invention is directed toward lightweight, inexpensive instruments that may be used for trace-level measurements of nitrogen oxides in, for example, atmospheric research, fields including medical research and diagnosis and vehicle exhaust measurement, and the like.

2. Description of Related Art

Previous gas-phase, trace-level measurements of nitrogen oxides have focused on the reaction of nitric oxide, NO, with ozone:

NO+O₃→NO₂*+O₂

The resulting nitrogen dioxide is initially formed in an excited state. This excited NO₂* may, under conditions of very low pressure, stabilize by releasing a photon, as is used in the common chemiluminescent nitric oxide sensors:

NO₂*→NO₂+hv

Alternatively, under atmospheric pressure conditions, the excited NO₂* is simply quenched by collisions with other molecules. In such a case, the decrease in ozone concentration may be measured and quantitatively related to the quantity of NO initially present, presuming accurate measurements of ozone concentration are made both prior to and after exposing the gas containing ozone to the sample air which contained the NO. The latter approach, herein referred to as the ozone depletion method, is described by Birks and Bollinger in U.S. Patent Application Publication 2004/0018630 A1, which is incorporated herein by reference in its entirety.

The chemiluminescent method depends upon providing a low-pressure region so that the excited NO₂* is not quenched but instead releases a photon; the attendant requirement for a vacuum chamber and vacuum pump adds significant weight and cost to these instruments. In addition, the chemiluminescent method requires calibration with expensive standards of NO gas. The ozone depletion method is susceptible to interference by any species which reacts with ozone, not just NO.

As a result, there is a need for a simple, lightweight, low-cost, and accurate instrument for the measurement of NO.

Scientific work over the past decade has demonstrated that the concentration of NO in human breath can be a good indicator of inflammation in the lungs caused by asthma and other respiratory diseases. Nitric oxide measurements can be made of expired air samples which provide a guide for diagnosis and treatment of inflammatory diseases in mammals, e.g., humans, and especially diseases involving the respiratory tract. A large body of medical literature has established a correlation between eosinophilic inflammation and elevated nitric oxide (NO) concentrations in human exhaled breath samples (above 30 parts per billion). Consequently it is now accepted by most academicians and clinicians that measurement of NO in exhaled breath can provide accurate, sensitive and immediate detection of eosinophilic inflammation in asthmatics. This biomarker can be used to diagnose asthma and manage asthmatics with anti-inflammatory therapies, such as with inhaled or oral steroids.

This technology for measuring nitric oxide could be applied to other biological systems such as for the blood system. In blood samples, nitric oxide can be disassociated from nitrolysated proteins such as from nitrolysated hemoglobin or from other serum proteins. Nitric oxide can also dissolve in fluid phase directly or as part of the composition of nitrites or nitrates. Using chemical or photochemical means, nitric oxide could be released as a gas from blood and then monitored using the technologies described in this patent application. A specific application of this in medicine would be to measure NO concentrations from blood samples in a patient with presumed sepsis. Elevated measurements could be used to make a presumptive diagnosis of sepsis before bacterial culture results confirm the diagnosis. Serial measurements of NO could monitor efficacy of therapy in this condition. It is also possible to use this technology in other medical conditions such as to measure nitric oxide from injured skin, i.e., burns, or severe dermatological inflammation. It is also possible to apply this technology to any organ system in which a sample of nitric oxide can be obtained. Very recently, NO concentrations were noted to be elevated in a patient who experienced anaphylaxis and had no history of asthma. The NO concentrations in exhaled breath samples decreased over time back to a normal range. Hence it is possible that NO concentrations in exhaled breath could be used to monitor severe anaphylaxis during a hospitalization.

### SUMMARY

The aforementioned and other needs are addressed by the various embodiments and configurations of the present invention. The present invention is generally directed to the measurement of selected substances in samples, particularly to measurements using photolytic reactions, in which a first compound photolyzes into one or more further compounds/elements. The concentration(s) of the first compound and/or further compounds/elements may be measured, directly or indirectly, by suitable techniques.

In a first embodiment of the present invention, a method is provided that includes the steps:
irradiating a sample to convert a selected first sample component into a second component and oxygen, the oxygen reacting with molecular oxygen in the sample to form ozone;
(b) second component reacting with ozone to reduce ozone;
(c) measuring an ozone concentration and/or a change in ozone concentration in response to step (a) and (b).

In a preferred application, the first sample component is nitrogen dioxide and the second component is nitric oxide. The first step is photolyzing NO₂ to create NO and oxygen atoms which substantially immediately react with molecular oxygen to form ozone. Then, after removal of the photolyzing radiation, the second step is to allow the NO and ozone to recombine to recreate NO₂. This photochemical pathway is substantially unique to the NO/NO₂ pair—few other gases will participate in a similar reaction sequence. Following is the reaction sequence for the NO/NO₂ pair:

NO₂+hv→NO+O

O+O₂→O₃

NO+O₃→NO₂+O₂

The exemplary methodology described herein can operate at very high ozone concentrations (and can indeed benefit from them)—high concentrations are needed for the NO plus ozone reaction to recreate NO₂ at a high rate. As the method relies on the detection of changes in ozone concentration values rather than absolute concentration values, it becomes possible to use a variety of instrumental methods to detect this small pulsed or modulated absorption signal (from the photolysis-produced ozone and subsequently decreased concentrations of ozone) on top of a large background absorption signal (from the supplied ozone).

In contrast, the ozone depletion method of Birks et al relies on accurate absolute measurements of total ozone concentration, which can lead to a susceptibility to errors as well as interference of gases, such as terpenes in the atmosphere which can combine with ozone. It also requires the use of ozone on the order of NO concentration. Furthermore at typical ozone concentrations used in this method, the NO and ozone reaction will be slow. As a result, the ozone depletion method by necessity may only operate at relatively low speeds in comparison to the described exemplary method. The method of Birks et al also does not allow for the direct measurement of nitrogen dioxide.

An exemplary embodiment of the invention combines a sample gas with another gas or gases, as necessary, to achieve a sample mixture containing the original sample gas, oxygen, and ozone prior to or upon entering an optical cell. The optical cell is illuminated with a pulsed light source of sufficient intensity to photolyze a fraction or all of the NO₂ present, yielding NO and O atoms, the latter immediately combining with ambient oxygen to form ozone. The NO recombines with the ozone to recreate NO₂. The cycle is then repeated any number of times in the course of a measurement. Ultraviolet light (or other non-ultraviolet wavelengths of electromagnetic radiation suitable for ozone measurement) from the pulsed photolysis source or another source is used in conjunction with a detector to measure the resulting pulsed changes in ozone concentration, which then are related back to the quantity of nitrogen oxides present.

A respiratory circuit can be integrated with the aforementioned NO/NO₂ system to measure the level of NO in an exhaled air stream from a mammal, e.g., human. This respiratory circuit, which satisfies American Thoracic Society (ATS) recommendations and/or FDA guidance, provides an expired air sample from a test subject, or in a manner consistent with the measurement objectives, such that the air sample is supplied to the analyzing device for measurement of NO and/or NO₂ concentration(s).

In addition, an exemplary embodiment of the invention, by operating in a pulsed mode, has a major sensitivity advantage in that pulsed or modulated signals can be retrieved, isolated, and amplified by instrumental means with a much higher signal-to-noise ratio than can be achieved with non-pulsed methods. This benefit of pulsing or chopping a signal is well-known and often used to isolate very small signals on top of large background signals. By operating in this rapid pulsed mode, the exemplary method can be insensitive to instrumental changes occurring over comparatively long timescales, such as lamp fluctuations, thermal expansion, ozone source fluctuations and the like.

Recalibration requirements are also reduced as the measurement is based on absorption of ultraviolet light by ozone, which can be quantitatively related to the concentration of ozone via the known absorption cross-section of ozone. The quantitative relationships between NO, NO₂, and ozone in the instrument lead to a system that can be self-calibrating, thereby eliminating the need for compressed gas cylinders containing standard concentrations of nitric oxide to support the routine use of the instrument.

There are significant advantages for the medical applications of the device described herein as the device addresses many of the issues that have caused competitive NO devices to be commercially unsuccessful. Some of the exemplary advantages of this nitric oxide device in medical applications include: inexpensive components, ease of calibration, and, if the device is used in the medical field, other than mouthpieces, the device has no disposable parts, such as expensive disposable sensors for measuring nitric oxide. Additionally, the instrument is unaffected by humidity, carbon dioxide, or other gases such as terpenes that can affect ozone depletion measurement of nitric oxide. The instrument can also be lightweight and portable.

Thus, an exemplary embodiment of the invention relates to a photochemical sensing system that enables the measurement of nitrogen dioxide and nitric oxide by photolyzing nitrogen dioxide to form oxygen atoms which combine with oxygen molecules to form ozone, and subsequently ozone recombining with nitric oxide to form nitrogen dioxide. Changes in ozone concentration are then measured as a surrogate for the nitrogen dioxide and nitric oxide. Any species which photolyzes to yield oxygen atoms may be measured by this technique. Additional specificity for nitrogen oxides is conferred by allowing the nitric oxide to react with the ozone to recreate the nitrogen dioxide. By periodically photolyzing the nitrogen dioxide (to form ozone), and then allowing the resulting nitric oxide to react with the ozone (thereby reducing ozone), a pulsed signal is obtained whose amplitude is proportional to the total amount of nitrogen dioxide and nitric oxide present.

A medical application of this instrument can be used to measure nitric oxide concentrations in expired air samples of a human that can assist in diagnosis of respiratory diseases (i.e., bronchial asthma) and also provide guidance in therapy decisions such as dosing and choice of medications.

Aspects of the invention also relate to a nitric oxide concentration measurement technique and related measurement device.

Aspects of the invention also relate to various detector assemblies that can be used to detect nitric oxide concentrations.

Aspects of the invention further relate to using a pulsed arc lamp to photolyze nitrogen dioxide to form oxygen atoms which combine with oxygen molecules to form ozone.

Still further aspects of the invention relate to using the disclosed methodology to detect and measure any species which photolyzes to yield oxygen atoms.

Additional aspects of the invention are directed toward the configuration of an optical cell for photolysis and measurement.

Additional aspects of the invention are directed toward the configuration of an optical cell for pulsed photolysis and measurement in conjunction with an optical chopper, a beamsplitter and/or one or more filters, such as neutral density filters.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The above-described embodiments and configurations are not exhaustive. As will be appreciated, other embodiments of the invention are possible utilizing, alone or in combination, one or more of the features set forth above or described in detail below. Additionally, these and other features and advantages of this invention are described in, or are apparent from, the following detailed description of the exemplary embodiments.

### BRIEF DESCRIPTION OF THE DRAWINGS

The exemplary embodiments of the invention will be described in detail, with reference to the following figures wherein:

FIG. 1 is a block diagram illustrating an exemplary gas detection system according to the invention;

FIG. 2 is a block diagram illustrating a second exemplary gas detection system according to the invention;

FIG. 3 is a detailed block diagram of a photolysis module according to a second exemplary embodiment of the invention;

FIG. 4 is a detailed block diagram of a photolysis module according to a third exemplary embodiment of the invention; and

FIG. 5 is a flowchart illustrating an exemplary method for photolyzing NO₂ according to this invention.

### DETAILED DESCRIPTION

The exemplary systems and methods of this invention will be described in relation to photolyzing one or more gases. However, to avoid unnecessarily obscuring the present invention, the following description omits well-known structures and devices that may be shown in block diagram form, are generally known or are otherwise summarized. For purposes of explanation, numerous specific details are set forth in order to provide a thorough understanding of the present invention. It should however be appreciated that the present invention may be practiced in a variety of ways beyond the specific detail set forth herein.

The term “module” as used herein refers to any known or later developed hardware, software, or combination of hardware and software that is capable of performing the functionality associated with that element. Also, while the invention is described in terms of exemplary embodiments, it should be appreciated that individual aspects of the invention can be separately claimed.

While the present invention is discussed with reference to nitric oxide and nitrogen dioxide measurement, it is to be understood that the principles of the invention can apply to other gas-phase components, particularly which can participate in a reversible reaction cycle. In such reversible reactions, photolysis decomposes a first compound into a second compound and an oxygen atom, which then combines with molecular oxygen to form ozone, a well known strong oxidant. The formed ozone then reacts with the second compound to reform the first compound and molecular oxygen. By measuring incremental increases or decreases in ozone concentration, the concentration of the first and second compounds may be deduced.

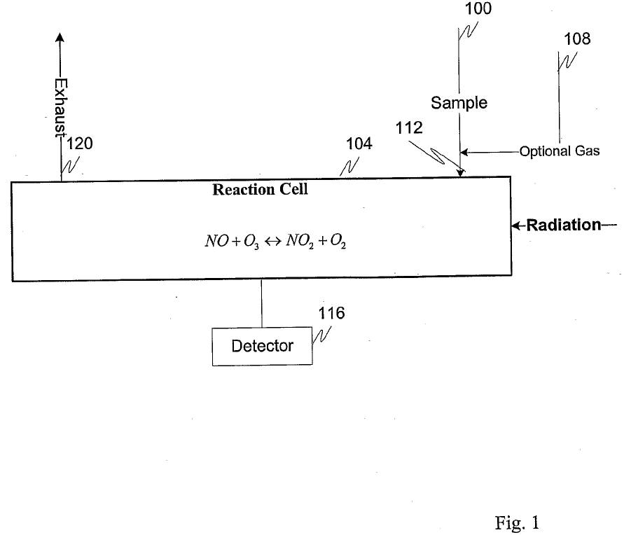

With specific reference to nitrogen oxides and with reference to FIG. 1, a sample gas 100 is introduced into a reaction cell or chamber 104, such as an optical cell. The sample gas 100 may have optionally been mixed with a second stream of ozone-bearing and oxygen-bearing gas 108 prior to entering the cell, or the mixing may occur in the cell 104 itself.

The compositions of the sample 100 and optional gas 108 depend on the application. The sample 100 and optional gas 108 are commonly substantially free of compounds, other than the target species that react with ozone before, during, or after radiation in the cell 104.

The resulting gas mixture may be flowing continuously through the cell 104, or may be stopped within the cell 104 during the measurement. In the course of the mixing, most preferably all ozone reactive species will react with ozone to form products. NO in the sample 100 will react with the ozone in the gas 108 to form NO₂ according to the following chemical equation.

NO+O₃→NO₂+O₂

NO₂ in the sample 100 is non-reactive with ozone and will therefore pass through the combination with the gas 108 unchanged. To ensure that substantially all of the NO in the sample 100 will be converted into NO₂, the gas 108 preferably contains a large excess of ozone to react with the NO in the sample 100. In this manner, the device can measure the total amount of nitrogen oxides (both NO and NO₂) in the sample 100, which is hereinafter referred to as NOₓ.

After the combined gas stream 112 is introduced into the reaction cell 104, a radiation source, such as pulsed light source, for example a pulsed xenon arc lamp, irradiates the cell with, for example, a rapid series of light pulses. The spectral output of the light source is selected so that some fraction of the light is capable of photolyzing NO₂ to yield NO plus oxygen atoms according to the following equation:

NO₂+hv→NO+O

Preferably, a sufficient number of light pulses is emitted so that a measurable amount of NO₂ in the cell 104 is converted into NO and ozone. The amount of energy required to yield this result depends, of course, on the amounts of nitrogen dioxide and other radiation absorbing molecules in the cell 104.

Oxygen atoms generated by nitrogen dioxide decomposition rapidly react with molecular oxygen to form ozone according the following equation:

O+O₂→O₃

This ozone will add to whatever concentration of ozone is initially present in the cell (larger amounts of originally present ozone will lead to faster reactions in the following steps, so it is expected that the instrument will supply a comparatively large amount of ozone in the gas 108). During irradiation, the reaction between NO and O atoms to reform NO₂ will occur at a completely negligible rate due to the very low concentrations of these two species and the very high concentration of molecular oxygen present.

After the photolysis pulse is done (or emission of radiation into the cell 104 terminated), the NO will immediately begin to react with the ozone (again, the ozone is provided in relatively large quantities relative to the small amount provided by the NO₂ photolysis to enable the following reaction to proceed at a rapid rate):

NO+O₃ [symbol] NO₂+O₂

The preceding series of three reactions adds up to a null reaction cycle, in which NO₂ is alternately destroyed during a radiation pulse and recreated between pulses. Ozone is measured in the instrument during pulses(s) or immediately after cessation of a pulse before a substantial amount of the ozone has had an opportunity to react with the NO to recreate the NO₂. As additional ozone forms only when NO₂ is photolyzed, and is destroyed only when NO is present, the modulated ozone absorption signal is directly proportional to the NO₂ concentration in the optical cell.

The ozone concentration in the cell 104 may be measured by any suitable technique, such as ultraviolet absorption during and/or after pulse emission. In a preferred configuration, the pulses include multiple radiation wavelengths, with one set of emitted wavelengths being absorbed by and effecting decomposition of the nitrogen dioxide while another set of disparate wavelengths is absorbed by the ozone. Ozone, for example, is known to absorb radiation having a wavelength of 253.7 nm and other wavelengths, particularly in the UV spectrum. In this manner, the same pulse effects both decomposition of the nitrogen dioxide and provides an optical signal that may be detected by an optical detector 116 to determine ozone concentration. The resulting, typically modulated, detector signal may then be measured for absolute amplitude differences between maximum and minimum levels or measured using a lock-in amplifier which isolates and amplifies the modulated component of the detector signal. The changes in the absorption signal may be quantitatively related to the changes in ozone concentration using the known absorption cross-section for ozone at the wavelength of the ultraviolet lamp. As there is a 1:1:1 stoichiometric relationship between ozone, NO and NO₂ via the preceding reactions, the ozone concentration changes found by this method equal the concentration of NO and/or NO₂ assuming the reactions go to completion; if they do not, calibration factors may be introduced to relate the two values. The absolute, relatively very high concentration of ozone does not need to be measured in that this high ozone concentration is manifested as a large background signal on top of which is the small modulated signal which is measured by this instrument.

After the measurement is completed, the combined gas 112 is removed from the cell 104 via the exhaust 120 and above steps repeated for a next combined gas 112. It is possible though to measure absolute absorbence from which the incremental changes may be calculated by differences.

As can be seen from this description, this method does not require any measurement or ozone concentration prior to or during this reaction. Although the total ozone concentration both before and after irradiation may be measured, it is preferred to measure only the change in ozone concentration. The change in ozone concentration is proportional to the amount of nitrogen dioxide and/or nitric oxide, or the total nitric oxide amount present in the cell 104.

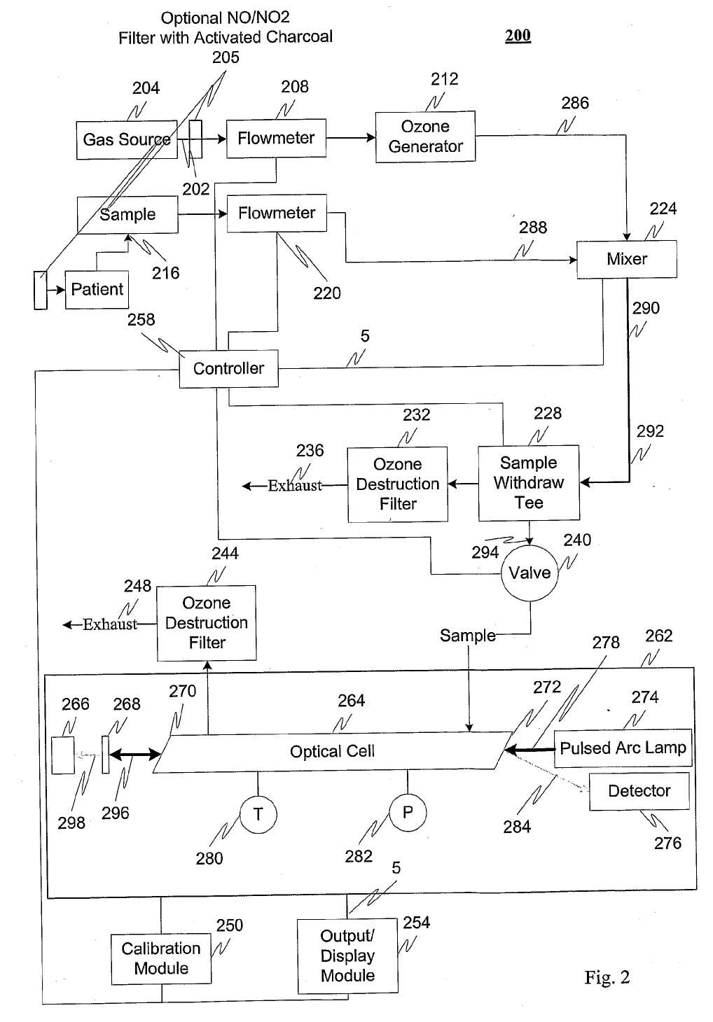

FIG. 2 illustrates an exemplary embodiment of the gas detection system 200. The system comprises a gas source 204, a flow-meter 208, an ozone generator 212, a gas sample source 216, a flow-meter 220, a mixer 224, a sample withdraw tee 228, an ozone destruction filter 232, an exhaust gas stream 236, a valve 240, an ozone destruction filter 244, another exhaust gas stream 248, a calibration module 250, an output/display module 254, a controller 258 and a photolysis module 262. The photolysis module 262 comprises a optical cell 264, a detector 266, a filter 268, such as a neutral density filter, optically transmissive windows 270 and 272, a pulsed arc lamp 274, second detector 276, main beam 278, temperature sensor 280, pressure sensor 282, and reflected beam 284, 296 (remaining and reflected portion of bean 278) and 298 (transmitted portion of beam 278).

The gas source 204, such as a pump or compressed gas cylinder, delivers a first stream of molecular oxygen-bearing gas into the gas detection system 200. The flow rate of the gas from the gas source 204 is measured with the flow-meter 208 for the purpose of achieving a reproducible flow rate amount relative to sample volume, with the gas from the gas source 204 entering the ozone generator 212, which produces ozone from the oxygen in the gas stream from the gas source 204.

A second stream of gas from the gas sample source 216 containing NO, NO₂, or a combination of gases to be measured, such as expired air from a human, enters the gas detection system 200, and the flow rate of the sample gas is measured at the second flow-meter 220.

Optional filters are shown upstream of the patient to treat the gas before it is inspired by the patient and between the gas source 204 and flow-meter 208 to treat the gas before it is passed through the ozone generator. The filters preferably remove at least most, more preferably at least about 90%, and even more preferably at least about 99% of the nitrogen oxides from the respective gas passing through each filter. In other words, the nitrogen oxides in the gas expired by the patient contains only nitrogen oxides generated in the respiratory tract of the patient while the gas 286 is substantially free of nitrogen oxides.

The first and second gas streams 286 and 288 are combined in the mixer 224 to form a combined gas stream 290, and the combined gas stream 290 then passed through a reaction area 292, such as a length of tubing, in which preferably substantially most and even more preferably at least about 95% of the NO in the second gas stream 288 reacts with ozone in the first gas stream 286 to form NO₂.

The combined gas stream 290 leaves the reaction area and enters the sample withdraw tee 228, from which a sample 294 of the combined gas stream 290 is periodically withdrawn via valve 240 in conjunction with, for example, controller 200.

The withdrawn sample 294 enters the optical cell 264. After a period of time during which the cell is flushed with sample 294, the valve 240 is closed to provide a stable cell environment for measurement. By closing the valve 240, pressure fluctuations can be eliminated which might otherwise enter the cell through the sampling tee. The balance of the gas stream 292 is continuously being dumped through the ozone destruction filter 232 (which destroys at least most of the ozone in the balance of the gas stream) and exits the instrument as exhaust stream 236. The gas sample 294 expelled from the optical cell 264 in the course of sampling exits the instrument through a second ozone destruction filter 244 as exhaust stream 248. As in the case of the balance of the gas stream, at least most of the ozone in the sample 294 is converted into molecular oxygen before discharge as the exhaust gas 248.

The measurement process begins by illuminating the optical cell 264 with a pulsed arc lamp 274, which emits ultraviolet radiation to encompass one or more wavelengths absorbed by ozone plus one or more wavelengths to effect nitrogen dioxide decomposition. An angled window 272 at the pulsed arc lamp side of the optical cell serves to deflect a small portion 284 of the radiation 278 into an optical detector 276. This measurement allows later correction for fluctuations in lamp power output in conjunction with the controller 258 and calibration module 250. The detector 276 may be preferably a photodiode paired with a 253.7-nm interference filter to allow selective detection of the 253.7-nm mercury line, so that the detector 276 is monitoring the same wavelength as detector 266.

The remainder, or primary, portion of this light beam 278 enters the optical cell 264 where the broadband emission of the lamp photolyzes substantially all of the NO₂ to yield NO and O atoms (which combine with molecular oxygen to produce ozone and the NO recombines with ozone to form NO₂ and molecular oxygen) and a portion of the radiation in the 253.7-nm emission is absorbed by ozone. The pressure 282 and temperature 280 sensors, in cooperation with controller 258 and calibration module 250, are mounted such that they may measure these variables within the cell to allow further correction of instrumental data.

The main beam 296, after passing through the optical cell 264, then exits the cell 264. The filter 268 attenuates the exiting light 296 with light reflected from the filter 268 (as shown by the double-headed arrow) re-entering the optical cell 264 to further contribute to the photolysis process. Preferably, the filter 268 is a reflective neutral density filter. In addition to enhancing photolysis, the filter 268 reduces the light intensity sufficiently that it may be measured by the detector 266 without destroying the detector or causing the detector 266 to operate in a nonlinear response region. Detector 266 is may be a photodiode paired with a 253.7-nm interference filter to allow selective detection of the 253.7-nm mercury line. This line is commonly used for absorption-based measurements of ozone and is used for that purpose in this instrument.

As the pulsed arc lamp 274 is pulsed over a period of time, ozone concentration will increase and decrease in the optical cell 264 in a pulsed fashion according to the chemistry described above. Detector 266 detects this ozone concentration increase as a reduction over time in the intensity of 253.7-nm light passing through the optical cell 264. The output of detector 266 may be divided, with the cooperation of controller 258, by the output from detector 276 to yield a signal, which is corrected for lamp intensity fluctuations. The uncorrected signal from the detector 266 may be used alone.

The signals from the detectors 266 and 276 are collected over time, and signal from detector 266 or the quotient of the output of detector 266 divided by output of detector 276 may be plotted, with the cooperation of controller 258, against time and output on output/display module 254. The resulting curve may be fit by or mapped against a selected set of mathematical equations to select a curve that fits a final set of parameters, which are directly related to the concentration of NOₓ species in the original sample.

As an alternative configuration of the gas detection system 200, the two incoming air streams from the gas source 204 and gas sample source 216 may be treated as one, and brought through the ozone generator 212. The exhaust streams 236 and 244 may also share a common ozone destruction filter. Furthermore, the entire gas stream exiting the reaction area 292 may be routed through the optical cell 264, provided pressure fluctuations are monitored and accounted for if necessary. Also, other types of optical filters may also be used, however, the greater the reflective properties, the greater the photolysis efficiency of the optical cell.

A pressure meter (not shown) could also be located between gas sample source 216 and flow-meter 220. The pressure meter could provide a means to measure back pressure that conforms to, for example, ATS recommended range since adequate back pressure creates expiratory resistance to allow closure of velum during expiratory air sampling from the patient, thereby minimizing contamination from upper airway nitric oxide. For example, the back pressure should be sufficient to close the vellum which is recommended by the ATS standard to be between 5 to 20 centimeters of water pressure.

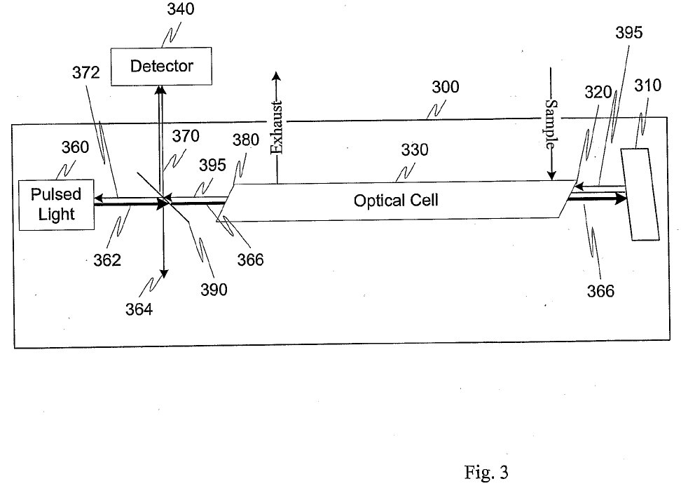

FIG. 3 illustrates a second exemplary embodiment of a photolysis module 300 for use in the gas detection system 200. Unlike the prior embodiments, this embodiment uses multiple radiation emitters and a common detector. One of the radiation sources, namely pulsed light source 360, generates pulsed first radiation to effect decomposition of nitrogen dioxide while the other radiation source, namely lamp 310, generates radiation substantially including radiation in the wavelength of 253.7 nm to measure absolute or relative ozone concentration.

With reference to FIG. 3, the photolysis module 300 comprises a second radiation source, or lamp 310, an optical cell 330, a beamsplitter 390, windows 320 and 380, a detector 340, and a first radiation source, or pulsed light 360. A sample is introduced to the optical cell 330 as described in relation to FIG. 1. The beamsplitter 390 is configured to allow the detector 340 to be protected from the output of the photolysis lamp (otherwise the detector could be destroyed, yield a noisy signal due to thermal noise, or be blinded by the photolysis beam). The beamsplitter 390 divides the radiation emitted by both the pulsed light 360 and lamp 310. Pulsed first radiation 362 is divided into two beams, 364 (which is lost) and 366 (which enters cell 330). Continuous second radiation 395 is divided into beam 370 (which goes to the detector 340) and beam 372 (which is lost).

The faces of the optically transmissive windows 320 and 380 may be angled so as not to return reflections of the first radiation 366 back to the beamsplitter 390 where the reflections could be bounced up to the detector 340. Preferably, none of the first radiation is directed to the detector 340, Whether by operation of the beamsplitter and/or reflections from the windows 380 or 320. Preferably, the window faces are inclined, relative to vertical, at an angle causing reflected beam to miss detector 340. The depicted optical arrangement reduces exposure of the detector to the first radiation (e.g., photolysis light) without resorting to the use of mechanical choppers or other mechanisms, and allows the detector 340 to observe the optical cell 330 throughout the photolysis sequence.

Between or during pulsed emissions of the first radiation 362, the lamp 310 (ideally a 253.7-nm mercury lamp) illuminates the optical cell 330 from the opposite direction relative to the first radiation 362. The beamsplitter 390 directs a first portion 372 of the second radiation 395 towards the pulsed light 360 and a second portion 370 towards detector 340. Detector 340 is functionally equivalent to detector 266 described earlier.

While pulsed arc lamps are described, a variety of photolysis light sources may be used including one or more of a pulsed arc lamp, a continuous arc lamp employed with an optical chopper, or a pulsed laser. Additionally, detectors may include photodiodes, phototubes, photomultiplier tubes, and other photosensitive detectors. The illustrated filters may be included with or external to the detector and the detectors may be inherently blind (e.g., “solar-blind”) to the photolysis lamp.

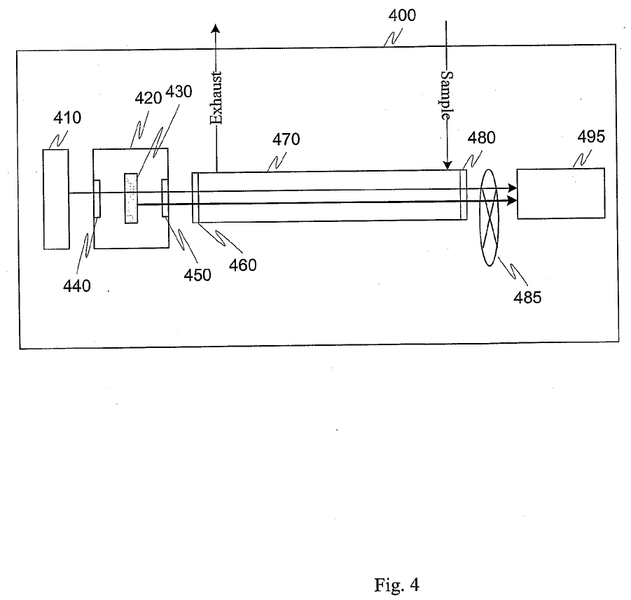

FIG. 4 represents a third exemplary embodiment of the photolysis module 400. The embodiment differs from the prior embodiments in that it includes first and second radiation sources positioned on a common end of the optical cell and a chopper.

The photolysis module 400 comprises a second radiation source or lamp 410, a first radiation source, or pulsed arc lamp 430, in a housing 420 with optically transmissive ports 440 and 450, an optical cell 470 with optically transmissive windows 460 and 480, an optical chopper 485, and a detector 495. A sample enters and exits the optical cell 470 as described in relation to FIG. 1. In this arrangement, radiation from the pulsed arc lamp 430 and lamp 410, such as a UV lamp, passes through the optical cell 470. The detector 495 is protected from the intense light of the pulsed arc lamp 430 by locating the detector 495 behind an optical chopper 485 at one end of the cell, with both radiation sources 410 and 430 being located at the other end of the optical cell. In one configuration, the radiation of the two radiation sources is combined by passing the first and second radiation through a beamsplitter (not shown) or by passing the second radiation of the lamp 410 through the body of the other radiation source (as shown). In this configuration the chopper physically blocks the radiation from the first radiation source 430 from reaching the detector 495 when the source 430 is fired. The chopper then rotates out of the way to allow light from second source 410 to be measured by the detector 495.

While the exemplary embodiments are described in relation to nitric oxide and nitrogen dioxide detection, other species may be detected that undergo photolysis to yield atomic oxygen plus a second species which can react with ozone to recreate the original species. The measurement may be carried out in any gas, provided that sufficient ozone and oxygen are provided to enable the described reaction sequences under the prevailing conditions.

If the sample is an oxygen-bearing gas mixture, i.e., air being one example, additional ozone may be generated directly from the oxygen in the sample gas mixture rather than in a separate gas stream. The absolute concentrations or changes of ozone may be measured while the photolysis lamp is on and off, and their difference used to compute the quantity of nitrogen oxides (e.g., NO₂ and NO), which are present in the irradiated sample. Systems specific for either NO or NO₂ may be also be created by selectively removing, via chemical or other means, the other species from the incoming gas stream. The ozone may also be measured by other methods, such as electrochemical methods, solid-state ozone sensors, or by optical absorption using non-ultraviolet wavelengths of light suitable for ozone measurement.

A respiratory circuit, designed to measure the level of NO in an exhaled air stream from a mammal, e.g., human, can be integrated to the aforementioned systems. A typical measurement of NO concentration in exhaled breath samples is referred to as the fractional concentration of nitric oxide in exhaled air (FENO) measured at a flow rate of 50 ml/sec according to American Thoracic Society recommendations. The fractional concentration of nitric oxide in exhaled air (FENO) can also be measured at higher flow rates but there is an inverse relationship between higher flow rates and FENO. As used herein, the term NO concentration can be interchangeably expressed as FENO at any flow rate used. Additionally the term NO concentration does not have to be restricted to fractional concentrations of NO as it could also represent total NO concentrations in exhaled human breath, depending on the objectives using the instrument. The respiratory circuit can receive exhaled air samples from a test subject in accordance with American Thoracic Society (ATS) recommendations and/or FDA guidance, or in a manner consistent with the measurement objectives, such that the air sample is supplied to the analyzing device for measurement of NO and/or NO₂ concentration(s).

The respiratory circuit can also have a configuration as described in the 2005 ATS/ERS recommendations for standardized procedures for the online measurement of exhaled lower respiratory nitric oxide (FIG. 1, page 915, thereof, which is incorporated herein by reference). A generic respiratory circuit device can include the following components: a mouthpiece means with a nitric oxide scrubber; valve system means in the respiratory circuit to permit inspired air to filter through a nitric oxide scrubber into the respiratory tract of test subject; means to measure back pressure in the ATS recommended range such that adequate back pressure creates expiratory resistance to allow closure of velum during expiratory effort, thereby minimizing contamination from upper airway nitric oxide; a valve means to permit test subject to exhale an air sample through mouth piece for access to NO analyzer; a means to monitor flow rates for specified time periods of exhalation; a means for data to be transferred and translated by a computer and a means for a numerical value read out of nitric oxide concentrations. It is possible for the respiratory circuit to be comprised of more or less components, providing, for example, the method for nitric oxide measurements conforms to ATS recommendations and/or FDA guidance.

As an alternative to measuring ozone as an indirect measurement of NO concentration, it is also possible to measure incremental increases and decreases of nitrogen dioxide and/or nitric oxide concentrations to reflect NO concentration. The detected signals of photochemically-modulated changes of NO or NO₂ concentrations would provide a means to measure NO concentration, using absorption of non-ultraviolet wavelengths of electromagnetic radiation specific for either gas.

Regarding nitric oxide, its concentration decreases as it combines with ozone and then increases after nitric dioxide is photolyzed by the light source; hence there is an incremental decrease and increase of nitric oxide in this application.

Regarding nitrogen dioxide, there is an incremental increase in its concentration after nitric oxide combines with ozone and then an incremental decrease in its concentration after photolysis of nitrogen dioxide to nitric oxide and oxygen atoms.

The incremental decrease and increase of nitric oxide concentration and/or incremental increase and decrease of nitrogen dioxide concentration could result in signals of photochemical modulated changes of NO or NO₂ respectively. These signals would provide a means to measure NO concentration using absorption of non-ultraviolet wavelengths of electromagnetic radiation specific for either gas. Since there is a 1:1:1 stoichiometric relationship between ozone, NO and NO₂ via the preceding reactions, the NO and/or NO₂ concentration changes by this method would equal the concentration of NO assuming the reactions go to completion; if they do not, calibration factors may be introduced to relate the two values.

Some de novo NO in the exhaled breath sample could be oxidized to NO₂ before the sample accesses the optical cell. Assuming NO and NO₂ are removed in the inspiration loop (ambient air breathed in through an activated carbon filter 205 as shown in FIG. 2), the NO concentration in an exhaled breath sample can be measured with the addition of a NO₂ removal filter in the exhalation loop. Conversely NO concentration can be measured in another exhaled breath sample from the same patient without the NO₂ removal filter in the exhalation loop. Measuring NO without the NO₂ filter would be higher than measuring NO with the NO₂ filter since in the former situation there will be extra NO₂ that was oxidized from de novo NO. In that scenario, there may be an increase in total NO₂ concentration in the optical cell, resulting in higher NO concentration. This can be important as current instruments may only be measuring a fraction of total NO that was in the exhalation sample since a portion of NO may be converted to NO₂.

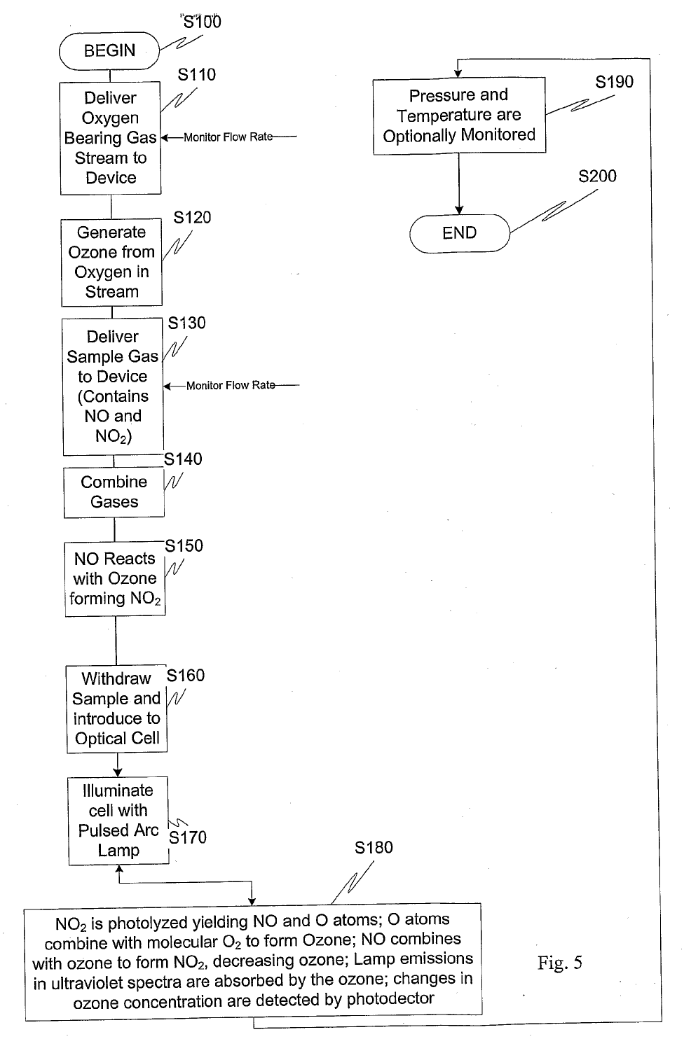

FIG. 5 illustrates an exemplary embodiment of detecting a selected gas component, such as NO, according to this invention. With reference to FIGS. 2 and 5, the process begins in step S100 and continues to step S110.

In step S110, molecular oxygen bearing gas 202 is delivered to the measurement device 200. In conjunction with the delivery of the oxygen bearing gas 202, the flow rate of the incoming gas stream 202 can be measured and monitored by flow meter 208. Next, in step S120, ozone is generated from molecular oxygen in the incoming gas stream 204. Then, in step S130, the sample gas 216 is delivered to the device 200. This sample gas contains NO and/or NO₂. In a similar manner to the oxygen bearing gas, the sample gas 216 flow rate can be monitored and measured by flow meter 220.

In step S140, the gases are combined in the mixer 224.

Next, in step S150, NO reacts in the reaction area 292 with the ozone to form NO₂.

In step S160, a sample 294 is withdrawn at sample withdraw tee 228 and introduced to the optical cell. The optical cell can optionally be sealed by valve 240 to reduce the pressure fluctuations therein.

In step S170, the optical cell 264 is illuminated with a pulsed arc lamp 274. In step S170, as the main beam 278 from the pulsed arc lamp exits the optical cell 264, and to optionally enhance photolysis, the neutral density filter 268 can be used to attenuate the main beam 296 for measurement and to reflect a reflected beam back into the optical cell.

In step S180, NO₂ is photolyzed yielding NO, O atoms, and additional ozone and the ozone recombine with NO to form NO₂ and molecular oxygen. Lamp emissions in the UV spectrum are being absorbed by the ozone and detected by the photo-detector.

In step S190, pressure and temperature can optionally be monitored within the optical cell by sensors 282 and 280, respectively.

While the above-described flowchart and methodologies have been discussed in relation to a particular sequence of events, it should be appreciated that changes to this sequence can occur without materially effecting the operation of the invention. Additionally, the exemplary techniques illustrated herein are not limited to the specifically illustrated embodiments but can also be utilized with the other exemplary embodiments and each described feature is individually and separately claimable.

Additionally, the systems and methods of this invention can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device such as PLD, PLA, FPGA, PAL, any comparable means, or the like. In general, any device(s) or means capable of implementing the methodology illustrated herein can be used to implement the various aspects of this invention.

Furthermore, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this invention is dependent on the speed and/or efficiency requirements of the system, the particular function, and the particular software or hardware systems or microprocessor or microcomputer systems being utilized.

Moreover, the disclosed methods may be partially implemented in software that can be stored on a storage medium, executed on programmed general-purpose computer with the cooperation of a controller and memory, a special purpose computer, a microprocessor, or the like. The system can also be implemented by physically incorporating the system and/or method into a software and/or hardware system, such as the hardware and software systems of a gas detector.

It is therefore apparent that there has been provided, in accordance with the present invention, systems and methods for detecting gas. While this invention has been described in conjunction with a number of embodiments, it is evident that many alternatives, modifications and variations would be or are apparent to those of ordinary skill in the applicable arts. Accordingly, the description is intended to embrace all such alternatives, modifications, equivalents and variations that are within the spirit and scope of this invention.

1. A system, comprising:
(a) a first radiation source operable to irradiate a gas sample to effect decomposition of a selected first sample component into a second component and oxygen atoms, the oxygen reacting with molecular oxygen in the gas sample to form ozone;
(b) the second component reacting with ozone to cause a decrease in ozone; and
(c) a first detector adapted to detect and/or measure ozone in the gas sample.

2. The system of claim 1, wherein the first detector measures at least one of an increase and decrease in ozone, wherein the selected first sample component is nitrogen dioxide, wherein the second component is nitric oxide, wherein, when the sample is irradiated by first radiation source with a first wavelength having sufficient energy to break nitrogen dioxide chemical bonds such that nitrogen dioxide is photolyzed to nitric oxide and oxygen atoms, which in turn recombine with molecular oxygen to form ozone and wherein nitric oxide decreases ozone by combining with ozone to form nitrogen dioxide and molecular oxygen.

3. The system of claim 2, wherein the gas sample is contacted with ozone before irradiation to convert substantially all nitric oxide in the sample into nitrogen dioxide.

4. The system of claim 1 wherein the first radiation source has a second wavelength which is absorbed by ozone and wherein the first radiation source emits both the radiation having the first and second wavelengths.

5. The system of claim 1, further comprising an optically transmissive window positioned between the gas sample and the radiation source, the window directing a portion of the radiation emitted by the radiation source to a second detector, and a neutral density filter positioned between the gas sample and the first detector to substantially attenuate radiation prior to contact of the radiation with the first detector.

6. The system of claim 1, further comprising a second radiation source, an optical filter, and beam splitter, the first and second radiation sources being positioned on opposing sides of the gas sample, wherein the first radiation source emits first radiation having at least a first wavelength with sufficient energy to break chemical bonds of nitrogen dioxide, wherein the second radiation source emits second radiation having at least a second wavelength to absorb ozone, wherein the first and second radiation are emitted over different periods of times, wherein the beam splitter is oriented so that a portion of the second radiation is directed to the first detector.

7. The system of claim 1, further comprising a second radiation source, an optical filter, and a chopper, the first and second radiation sources being positioned on a common side of the gas sample, wherein the first radiation source emits first radiation having at least a first wavelength with sufficient energy to break chemical bonds of nitrogen dioxide, wherein the second radiation source emits second radiation having at least a second wavelength to absorb ozone, wherein the first and second radiation are emitted over different periods of times, wherein the chopper is oriented to block at least a portion of the first radiation from contacting the first detector.

8. The system of claim 1, wherein first radiation emitted by the first radiation source comprises non-ultraviolet radiation wavelengths, wherein a portion of the first radiation, after exiting the gas sample, is directed back through the gas sample to contribute further to decomposition of the first sample component,

9. The system of claim 1 which does not require recalibration, wherein the system further comprises a respiratory circuit to provide the gas sample, and wherein the first detector is one of a solid-state ozone sensor, a radiation sensor, and an electrochemical sensor.

10. A system adapted to measure one or more of nitric oxide and nitrogen dioxide in a sample using radiation-induced changes of ozone concentration.

11. The system of claim 10, wherein the sample is a gas sample and further comprising:
(a) a first radiation source with first wavelength operable to irradiate the sample to effect decomposition of nitrogen dioxide into nitric oxide and oxygen atoms, the oxygen reacting with molecular oxygen in the sample to form ozone;
(b) the nitric oxide reacting with ozone to decrease ozone; and
(c) a first detector adapted to detect and/or measure at least one of an increase and decrease in ozone, nitric oxide, and nitrogen dioxide in the sample.

12. The system of claim 11, wherein the first detector measures ozone and further comprising a controller adapted to correlate radiation changes in ozone concentration to concentrations of nitric oxide and/or nitrogen dioxide in the sample gas.

13. The system of claim 11, wherein the gas sample is a gas expired by a patient.

14. The system of claim 11, wherein the gas sample is contacted with ozone before irradiation to convert substantially all nitric oxide in the sample into nitrogen dioxide, wherein the radiation has a second wavelength absorbed by ozone and wherein a common radiation source emits radiation having the first and second wavelengths.

15. The system of claim 11, further comprising an optically transmissive window positioned between the gas sample and the radiation source, the window directing a portion of the radiation emitted by the radiation source to a second detector and a neutral density filter positioned between the gas sample and the first detector to attenuate substantially radiation prior to contact of the radiation with the first detector.

16. The system of claim 11, further comprising a second radiation source, an optical filter, and beam splitter, the first and second radiation sources being positioned on opposing sides of the gas sample, wherein the first radiation source emits first radiation having at least a first wavelength capable of breaking nitrogen dioxide chemical bonds, wherein the second radiation source emits second radiation having at least a second wavelength absorbed by ozone, wherein the first and second radiation are emitted over different periods of times, wherein the beam splitter is oriented so that a portion of the second radiation is directed to the first detector.

17. The system of claim 11, further comprising a second radiation source, an optical filter, and a chopper, the first and second radiation sources being positioned on a common side of the gas sample, wherein the first radiation source emits first radiation having at least a first wavelength capable of breaking nitrogen dioxide chemical bonds, wherein the second radiation source emits second radiation having at least a second wavelength absorbed by ozone, wherein the first and second radiation are emitted over different periods of times, wherein the chopper is oriented to block at least a portion of the first radiation from contacting the first detector.

18. The system of claim 11, further comprising a radiation source and detector, wherein first radiation emitted by the radiation source comprises non-ultraviolet radiation wavelengths, wherein a portion of the radiation, after exiting the sample, is directed back through the sample to contribute further to decomposition of the nitric dioxide, wherein the system does not require recalibration, wherein the sample is a gas sample, wherein the system further comprises a respiratory circuit to provide the gas sample, and wherein the detector is one of a solid-state ozone sensor, a radiation sensor, and an electrochemical sensor.

19. A method, comprising:
(a) irradiating a sample to convert a selected first sample component into a second component and oxygen atoms, the oxygen reacting with molecular oxygen in the sample to form ozone;
(b) the second component reacting with ozone to cause a decrease in ozone; and
(c) measuring at least one of a concentration and a change in concentration of ozone, nitric monoxide, and nitric dioxide after step (b).

20. The method of claim 19, wherein the sample is a gas, wherein first sample component is nitrogen dioxide, wherein the second component is nitric oxide, wherein:
(a) during irradiation of the sample, the first sample component is photolyzed to the second component and oxygen atoms, wherein oxygen atoms combine with molecular oxygen to form ozone, wherein ozone combines with second component to form first component causing a decrease in ozone;
(b) wherein, in the measuring step, at least one of a concentration and change in concentration of ozone is measured by ultraviolet absorption of ozone in a detector; and
(c) based on the measurement of step (b), determining at least one of concentration of and a change in concentration of the first sample component and/or second component.

21. The method of claim 19, further comprising:
(a) receiving the sample;
(b) contacting the sample with a gas comprising ozone and molecular oxygen to form a mixture of the sample and gas, introducing a portion of the mixture into a cell, wherein, in the irradiating step and while the mixture is in the cell, the mixture is irradiated with an intermittent radiation sufficient to photolyze nitrogen dioxide, wherein the photolytic reaction forms nitric oxide and oxygen atoms, the oxygen atoms then reacting with ambient molecular oxygen to form ozone, and then ozone combines with nitric oxide to form nitrogen dioxide causing a decrease in ozone; and
(c) wherein the determination is made by measuring absorption of ozone by ultraviolet light in a detector.

22. The method of claim 20, wherein a first portion of the radiation is directed to a first detector and a second portion is directed to the cell, and further comprising:
after the second portion of the radiation has passed through the cell, attenuating the second portion of the radiation; and
contacting the attenuated second portion with a second detector.

23. The method of claim 21, wherein the irradiating step is performed by a first radiation source and wherein the determining step comprises the sub-steps:
a second radiation source irradiating the mixture with second radiation, a portion of the second radiation being absorbed by ozone in the mixture;
after the second radiation exits the cell, directing a first portion of the second radiation to a detector and a second portion of the second radiation away from the detector;
passing the first portion of the second radiation to a detector; and
, wherein the first and second radiation are emitted over different periods of times.

24. The method of claim 21, wherein the irradiating step is performed by a first radiation source and wherein the determining step comprises the sub-steps:
a second radiation source irradiating the mixture with second radiation;
and wherein the first and second radiation are emitted over different periods of times.

25. The method of claim 21, wherein the sample gas is obtained from a biological system capable of providing said sample gas and wherein said biological system is blood.

26. The method of claim 21, wherein the sample gas is obtained from a biological system capable of providing said sample gas and wherein said biological system is skin.

27. The method of claim 21, wherein the sample gas is obtained from a biological system capable of providing said sample gas and wherein said biological system is the lung, such that measurement of NO concentration in exhaled breath samples during anaphylaxis is used as a biomarker of anaphylaxis to support diagnosis of anaphylaxis and to monitor treatment of anaphylaxis.

28. The method of claim 21, wherein at least one of an increase and decrease in ozone concentration is measured, wherein, in the irradiating step, a portion of the radiation, after passing through the sample, is redirected back through the sample to contribute further to photolysis of the first sample component, and wherein a common radiation source provides radiation having a first wavelength to photolyze the first sample component and to measure the ozone.

29. A method to determine NO in a breath sample from a human, said method comprising:
(a) exhaling breath sample through a respiratory circuit into a NO analyzer;
b) said analyzer capable of measuring photochemically modulated changes of ozone concentration in said sample;
c) said changes of ozone concentration converted into concentration of NO in said air ample.

30. The method of claim 29 wherein the breath sample is comprised of inspired air filtered through a NO filter.

31. The method of claim 29 wherein the expired breath sample courses through a nitrogen dioxide filter prior to entering said analyzer.
